(19) United States
(12) Patent Application Publication
(10) Pub. No.: US 2009/0285917 A1
(43) Pub. Date: Nov. 19, 2009

(54) COMPOSITION COMPRISING COMPLEX CRUDE DRUG EXTRACTS SHOWING ANTI-ALLERGIC RHINITIS, ANTI-ATOPIC DERMATITIS AND ANTI-ASTHMA ACTIVITY

(76) Inventors: Jong Moon Jeong, Gyeonggi-do (KR); Seung Sook Lee, Gyeonggi-do (KR); Kyung Bum Kim, Gyeonggi-do (KR); Eu Gene Lee, Gyeonggi-do (KR)

Correspondence Address: BERLINER & ASSOCIATES, 555 WEST FIFTH STREET, 31ST FLOOR, LOS ANGELES, CA, 90013, US

(21) Appl. No.: 12/441,689

(22) PCT Filed: Sep. 27, 2006
(86) PCT No.: PCT/KR2006/003856

§ 371 Date: Jul. 21, 2009

(30) Foreign Application Priority Data
Sep. 25, 2006 (KR) 10-2006-0092694

(51) Int. Cl. A61K 36/282; A61K 36/87; A61K 36/60; A61P 37/08; A61P 11/06; A61P 17/00

(52) U.S. Cl. 424/740; 424/766; 424/774

(57) ABSTRACT

A prevention and therapy pharmacy composition for allergic rhinitis, atopic dermatitis and asthma includes at least one crude drug extract selected from the group including wormwood, guava leaves, mulberry tree leaves and grape seeds as an effective component. The functional composition may be used for manufacturing a medicine composition and health function food with its anti-allergic rhinitis, anti-atopic dermatitis and anti-asthma activity effects.

### TECHNICAL FIELD

The present invention relates to a composition having a combined crude drug extract of wormwood, guava leaves and leaves of mulberry tree as an effective component having anti-allergic rhinitis, anti-atopic dermatitis and anti-asthma activity effects.

### BACKGROUND ART

A conventional anti-inflammation anodyne, which is generally used for allergic rhinitis, atopic dermatitis and chronic asthma, has an anti-inflammation anodyne effect by inhibiting a prostaglandins creation metabolism through an enzyme being called as lipoxygenase (LO) catalyzing the formation of leukotrienes, which causes a bronchus contraction, a bronchus over reaction, and an inflammation of an airway by metabolizing arachidonic acid, and an enzyme which is called as cyclooxygenase(COX) of cells. Here, the COX-1 is related to creating prostaglandins at inflammation portions as well as normal organs and tissue, namely, stomach tube or kidney. However, COX-2 is an enzyme which is related to a portion having an inflammation. Nonsteroidal anti-inflammatory drugs (NSAIDs) commercially available in the market inhibits COX-1 and COX-2 or mainly inhibits COX-1, so that it creates prostaglandins needed for maintaining inherent functions of stomach tubes or kidneys when it is taken for a long time period for thereby causing many side effects. (Isselbcher et al., Harrison's Principles of Internal Medicine(13th ed), 2, pp 1543-1711, 1994). The recently developed selective COX-2 inhibitor is able to largely decrease the side effects while keeping a known COX-2 anti-inflammation effect, so that the use of the same increases. So, the materials having a LO inhibition activation and selective COX-2 inhibition activation may be used for curing allergic rhinitis, atopic dermatitis and chronic asthma.

The mast cell widely spread in the internal engaging tissue had a function of producing materials contained in the cells when it is stimulated. These cells are used for developing a chemical able to prevent or cure various allergic diseases such as allergic rhinitis, atopic dermatitis and chronic asthma through the researches on de-granulation factors and inhibition factors. The induction mechanism on allergic diseases caused by the activations of mast cell and other cell is not known, but for curing the same, anti-histamine or steroid agent is generally used for releasing symptom.

In particular, histamine is generally stored in mast cell and basophil of a human as medium causing the allergic diseases. So, it is possible to develop an effectiveness of allergic rhinitis, atopic dermatitis and chronic asthma and medicine mechanism using the mast cells.

The effectiveness as a therapy agent of allergic rhinitis, atopic dermatitis and chronic asthma is studied through an actual animal test and an animal toxicity test.

Artemisia. Princes var. orientalis(PAMPAN) HARA is perennation plant and is made by drying the leaves of yellow sea wormwood and green plants of same group. Yellow sea wormwood contains refined oil of which most contained components is cineole while occupying 25-30%. In addition, there are β-carophyllene, linalool, camphor, borneol. The leaves contain tetracosanol, β-sitosterol, l-chebulachitol, l-inositol. Roots and stem contain artemose similar with inulin. Roots contains many kinds of polyin compounds and contains (heptadec-1,7,9,-trien-11, 13, 15-triyne), (tetradeca-8, 10, 12,-trine-6-ene-3-one) and (methyl 2-decen-4,6,8-triynate). It also contains a material operating as oxytoxin. Many plants include ridentin of sesquiterpene lactone. In addition, it is known as having a function of treating acupuncture spot and removing cold and providing blood warming effect, bleeding stop and pregnancy care. (Oriental medicine dictionary by Jung Bo-sup and Shin Min-kyo, Youngrim publication company, pp 1014-1016, 1998).

Morus alba Linn is the leaves of mulberry tree and contains rutin, quercetin, iso-quercetin, moracetin, a small amount of β-sitosterol, campesterol, lupeol, inkosterone, myoinositol, hemolysin. In addition, the refined component of the same includes acetic acid, propion acid, valerate, and capron acid. It is known as having hypertension curing function and anti-diabetes. (Medicine and plant dictionary by Park Jong-hee and Lee Jung-hee, Shinil corporation, 2000).

Psidium guajava L has a large amount of tannin which is known as having a strengthening function of stomach and intestines, and an aging prevention and anti-cancer functions. As other components, it contains vitamins and mineral such as fat oil, refined oil, gasoline, insulin component, vitamin B-group, vitamin C, magnesium, potassium. (Korean plants dictionary by Ahn Duk-kyun, Kyohak compony, 1999).

The grape seeds are known as strengthening moisture ratio of bone and blood vessels in Bonchogangmok of Lee Seo-jin, and provides patience with respect to fatigue and is good for colds. When a person takes it for a long time period, the person may feel lightness and fresh, so that the person can live for a long time.

There are many studies for developing a functional food or therapy agent using the extracts of the grape seed with its excellent biological effects. The Korean patent publication number 98-51189 discloses a grape seed as yrosinase inhibitor, and the Korean patent registration number 00-18117 discloses a grape seed extraction natural anti-insect agent, and the Korean patent publication number 01-12238 discloses a functional food containing the extracts of the grape seed such as rice coated with the extracts of the grape seed. The Korean patent publication number 00-63265 discloses a grape seed oil and a method for manufacturing the same having an anti-oxidation material, and the Korean patent publication number 01-04553 discloses a method for manufacturing a grape product having an enhanced anti-oxidation effect, and the Korean patent publication number 99-288d77 discloses the use of anti-oxidation effects of proanthocyanidin which is a main component of the grape seed extract such as chemical agent having procyanidin as an affective component.

Namely, wormwood, guava leaves, mulberry tree leaves and grape seed which have been used from a long time ago without toxicities and side effects in the oriental medicine field and residents are studied and researched with respect to their effectiveness and toxicity on scientific basis.

However, the books and researches on anti-allergic rhinitis, anti-atopic dermatitis and anti-asthma functional compositions containing combined crude extracts are not disclosed in any search reports and other data, and the researches on the same are not conducted ever.

The inventor of the present invention has conducted the foods on anti-allergic rhinitis, anti-atopic dermatitis and anti-asthma based on natural substances and has confirmed that the composition containing wormwood, guava leaves, mulberry tree leaves and grape seed extracts have anti-allergic rhinitis, anti-atopic dermatitis and anti-asthma activity effects.

### DISCLOSURE OF INVENTION

### Technical Problem

Accordingly, it is an object of the present invention to provide a composition having anti-allergic rhinitis, anti-atopic dermatitis and anti-asthma activity effects by using a combined crude extract of wormwood, guava leaves and grape seed.

### Technical Solution

To achieve the above objects, there is provided a prevention and therapy pharmacy composition for allergic rhinitis, atopic dermatitis and chronic asthma which includes at least one crude drug extract selected from the group including wormwood, guava leaves, mulberry tree leaves and grape seeds as an effective component.

To achieve the above objects, there is provided a prevention and therapy pharmacy composition for allergic rhinitis, atopic dermatitis and chronic asthma which includes a combined crude drug extract formed of wormwood, guava leaves, mulberry tree leaves and grape seeds.

In detail, the composition of the present invention has a mixing ratio of 1˜3 weight % of wormwood, 1˜2 weight % of mulberry tree leaves, 1˜2 weight % of guava leaves, and 2˜4 weight % of grape seeds.

The composition of the present invention is formed of wormwood water soluble extract of 5˜50 weight %, guava leaves water soluble extract of 10˜100 weight %, mulberry leaves water soluble extract of 5˜50 weight %, and grape seed water soluble extract of 5˜70 weight %, and more preferably, it is formed of wormwood water soluble extract of 10˜40 weight %, guava leaves water soluble extract of 20˜70 weight %, mulberry leaves water soluble extract of 10˜40 weight %, and grape seed water soluble extract of 10˜50 weight %.

### ADVANTAGEOUS EFFECTS

In the present invention, the functional composition of the present invention may be used for manufacturing a medicine composition and health function food with its anti-allergic rhinitis, anti-atopic dermatitis and anti-asthma activity effects.

### BRIEF DESCRIPTION OF THE DRAWINGS

FIG. 1 is a view of a toxicity test result with respect to a white rat abdomen mast cell at various concentrations of a functional composition of a second example of the present invention.

FIG. 2 is a view of an inhibition effect of a functional composition of a second example of the present invention with respect to histamine obtained from a mast cell with a compound 48/80 (allergic induction material).

FIG. 3 is a view of an inhibition effect of a functional composition of a second example of the present invention with respect to a mast cell degranulation with a compound 48/80.

### BEST MODE FOR CARRYING OUT THE INVENTION

In the present invention, a prevention and therapy pharmacy composition for allergic rhinitis, atopic dermatitis and asthma includes at least one crude drug extract selected from the group including wormwood, guava leaves, mulberry tree leaves and grape seeds as an effective component.

### MODE FOR THE INVENTION

The present invention will be described in details.

In order to manufacture the extracts of wormwood, guava leaves and mulberry tree leaves used in the present invention, wormwood, guava leaves, mulberry tree leaves and grape seeds are washed, and dried under shadow and are cut and ground and are processed in water of about 1 through 30 times of the weight(kg) or a low grade alcohol or a mixed solvent of the same at 20 through 100° C., preferably at an extraction temperature of 80 through 100° C. for about 1 hour or 2 days, preferably, about 2 hours through 12 hours and for one time or 10 times, preferably 2 through 5 times, based on a heat water extraction method, a ultrasonic wave extraction method, or a circulation cooling extraction method, and more preferably, 80 through 110° C. for 1 through 5 hours for thereby obtaining a combined crude drug extract of the present invention. It may be filtered and condensed with a pressure down concentration method for thereby obtaining an extraction condensation liquid, and it may be frozen and processed in powder.

The present invention provides a method for manufacturing a combined crude drug extract of wormwood, guava leaves, mulberry tree leaves and grape seed.

In addition, the present invention provides a functional composition including a combined crude drug extract of wormwood, guava leaves and mulberry tree leaves which are effective to anti-allergic rhinitis, anti-atopic dermatitis and anti-asthma activity.

The crude drug extract has been used for a long time, and the extracts of the present invention does not have toxicity and side effects.

As a result of the animal test after the inhibition effect, mast cell activation inhibition effect of lipoxygenase and cyclooxygenase enzyme, which causes an inflammation of the composition obtained by the above method are performed, it is known that anti-allergic rhinitis, anti-atopic dermatitis and anti-asthma activity effects are excellent.

The pharmacy composition effective to allergic rhinitis, atopic dermatitis and asthma activity includes the combined crude drug extract by 0.1 through 50 weight % with respect to the total weight of the composition.

The pharmacy composition including the composition of the present invention includes a carrier, Excipients and diluents.

The pharmacy composition including the composition of the present invention may be provided in the form of oral administration types such as acids, granule, tablet, capsule agent, supernatant, emulsion, syrup, aerosol, external administration, Suppository and disinfections injection solution. The carrier, excipients and diluents of the composition of the present invention includes lactose, dextrose, sucrose, sorbitol, mannitol, xylitol, erythritol, Maltitol, starch, acacia rubber, alginate, gelatin, calcium phosphate, calcium silicate, cellulose, methyl cellulose, non-crystalloid cellulose, polyvinyl Pyrrolidone, water, methyl hydroxyl benzoate, propylhydroxy benzoate, talc, magnecium Stearate and minerals. They are generally formulated by using a diluents or excipients such as filler, binder, moisturizer, disintegrant, and diluents or shaper such as surface active agent. The solid agents for oral administration include tablet, circular agent, acids, granule, and capsule agent. The above solid agent is manufactured by mixing at least one excipients with the above composition such as starch, calcium carbonate, sucrose or lactose, gelatin, etc. Instead a simple excipient, the liquid agent includes supernatant, liquid agent, milky agent, syrup. It may include various recipients such as moisturizer, tasters, aromatic, storing aid, instead simple diluents of water, liquid paraffin, etc. The agent for non-oral administration includes disinfected solution, non-water soluble solvent, supernatant, milky agent, frozen and dried agent, Suppository. The non-water soluble suspension includes propylene glycol, polyethylene glycol, plant oil such as olive oil and injection ester such as ethlyoleate. The base agent of Suppository includes witepsol, Macrogol, tween 61, Cacaco butter, Laurin butter, glycerogelatin.

The amount of the administration of the composition of the present invention differ based on the state and weight of a patient, a disease state, a chemical type, and an administration passage and period, which may be properly selected by a person skilled in the art. Preferably, it is 0.001 through 100 mg/kg. The administration is provided one time a day, and may be provided a few times a day. The amount of the administration is not limited to the scope of the present invention.

The composition of the present invention may be administrated to mammal such as rat, mouse, livestock and human in various methods. All kinds of administration may be possible such as oral administration, rectum or vein, muscle, subcutaneous, intrauterine gastric or intracerebroventricular injection method.

Since the composition of the present invention does not have any toxicity and side effects, it is possible to intake for a long time for the purpose of prevention.

The present invention includes an effective component of crude drug extracts formed by selecting at leas one from the group comprising wormwood, guava leaves, mulberry tree leaves and grape seeds for thereby achieving a prevention and health improvement of allergic rhinitis, atopic dermatitis and asthma activity.

The health function foods of the present invention includes a ground substance, an extract or a powder type made from crude drug extracts.

The composition of the present invention may be used in drug, food and beverage for a prevention and improvement of allergic rhinitis, atopic dermatitis and asthma activity. The composition of the present invention may be included into various foods such as beverage, gum, tea, combined vitamin pill, and health supplemental food and may be used in the form of pill, powder, granular form, tablet, capsule or in the form of beverage. At this time, the amount of the composition of the health beverage is 0.02 through 5 g with respect to 100 ml, and preferably it is 0.1 through 1 g.

The composition of the health functional beverage of the present invention includes the above compositions as necessary components, and does not have any limitation with respect to the other components and may be added with various components such as tasters or natural carbohydrate etc.

The above natural carbohydrate includes glucose, fructose; disaccharide for example maltose, sucrose; polysaccharide for example, textrin, cyclodextrin, and sugar alcohol such as dilaritol, sorbitol, aerytritol. As aromatic except for the above elements, there are glucose, fructose; disaccharide, for example maltose, sucrose; and polysaccharide, for example dextrin, sugar such as cyclodextrin, and sugar alcohol such as xylitol, sorbitol, aerytritol. The ratio of the natural carbohydrate is about 1 through 20 g per 100 ml of the composition of the present invention, preferably it is about 5 through 12 g.

The composition of the present invention may include various nutrition, vitamin, mineral(electrolyte), synthesized tasters and natural tasters, coloring agent, and cheese and chocolate, pectic acid and salt of the same, alginic acid and salt of the same, organic acid, protective colloid thickener, pH adjuster, stabilizer, antiseptic agent, glycerin, alcohol, and carbonator used in carbonated beverage. The composition of the present invention may include flesh for manufacturing natural fruit juice and fruit juice beverage and vegetable beverage. The ratio of the above additive is not important, but it is preferably in a scope of 0g through about 20g per 100 weight % of the composition of the present invention.

The examples and tests of the present invention will be described in detail.

The examples and tests of the present invention are provided for only the illustrative purpose, and the contents of the present invention are not limited to the following example and tests.

### Example 1

### Preparation of Single Plant Extract

Wormwood, mulberry tree leaves and grape seed obtained by Nongrim crude drug corporation (Hoseo university TBI 114 of San 29-1, Sachil-ri, Baebang-myeon, Chungnam, Korea) and guava leaves obtained by Jingyong natural corporation (Vellocity 913, Ingae-dong 1135-1, Paldal-gu, Suwon city, Kyunggi-do, Korea) are heat-extracted by inputting by 1 kg into water of 5 L at 100° C. for 3 hours, and are condensed using a vacuum concentration unit(Buchi R114, Buchi corporation, Switzerland) until the solid component is concentrated and becomes 30%, and the condensed contents are frozen and dried using a freezing and drier(FD 5512, Ilsin company, Korea), and a dried powder of 82.5 g, 92.4 g, 102.3 g and 33.0 g is obtained from wormwood, mulberry tree leaves, grape seeds and guava leaves.

### Example 2

### Preparation of Functional Combined Crude Drug Composition

2-1. Preparation(1) of Functional Composition

The water soluble extracts of wormwood, guava leaves, mulberry tree leaves and grape seed of the example 1 are combined at a ratio of 9 g, 25 g, 8 g and 8 g with respect to the weight of the solid components, and a functional composition of the present invention is prepared.

2-2. Preparation(2) of Functional Composition

The water soluble extracts of wormwood, guava leaves, mulberry tree leaves and grape seed of the example 1 are combined at a ratio of 15 g, 20 g, 8 g and 7 g with respect to the weight of the solid components, and a functional composition of the present invention is prepared.

Test 1. Lipoxygenase Enzyme Inhibition Effect Measurement of Each Plant Extract

In order to check the anti-allergic rhinitis, anti-atopic dermatitis and anti-asthma activity effects with respect to each plant extract of the example 1 of the present invention, the lipoxygenase inhibition effects which is an inflammation and allergic induction enzyme are tested by changing the method disclosed in Hyo-Jin Kim et al J. Food Sci, Natr 3(3) pp 216-220, 1998.

Each plant extract of 20 ul and 200 unit enzyme lipoxygenase type V, L-6632, Sigma, USA of 10 ul was mixed with 1 ml of 0.1M tris buffer, Ph. 8.5, and was reacted at a room temperature for 5 minutes, and 30 ul of linoleic acid, 50 ug was added, and then the initial reaction speed was measured at 234 nm, and the IC₅₀ value was calculated using the formula 1.

LO inhibition activation(%)=((control group A−control group B)−(sample A−sample B)/(control group A−control group B))×100 [Formula 1]

control group A: initial reaction speed of control group not added with sample

Sample A: initial reaction speed of reaction group not added with sample

control group B: initial reaction speed of control group not added with linoleic acid

Sample B: initial reaction speed of reaction group not added with linoleic acid

As a result of the above tests, the IC₅₀ value of LO based on each extraction powder was 52.1 ppm, 14.1 ppm, 56.4 ppm, and 49.1 ppm(refer to Table 1).

TABLE 1

| Samples | Lipoxygenase IC50 (ppm) |
|---|---|
| Wormwood extract powder | 52.1 ppm |
| Guava leaves extract powder | 14.1 ppm |
| Mulberry tree leaves | 56.4 ppm |
| extract powder | |
| Grape seed extract powder | 49.1 ppm |

Test 2. Lipoxygenase Enzyme Inhibition Effect Measurement of Functional Composition

The functional composition of the example 2-1 of the present invention was tested for the LO inhibition activation test in the same method as the test 1. As the both control groups, EGCG((−)-epigallocatechine-3-gallate)(E4143, Sigma corporation, USA), which is effective component of green tea, was compared together.

As a result of the test, the inhibition activation IC₅₀ value of the functional composition of the example 2-1 with respect to LO was 11.2 ppm, and LO was inhibited by above 90% in 32 ppm. In addition, when it was compared with the EGCG used at the both control groups, it had a 7.6 times excellent effect(refer to Table 2).

TABLE 2

| Sample | Lipoxygenase IC50(ppm) |
|---|---|
| Functional composition | 11.2 |
| EGCG | 85.4 |

Test 3. COX-1, COX-2 Enzyme Inhibition Effect Measurement

In order to confirm the inhibition effects with respect to COX-1, COX-2 which are inflammation and allergic induction enzyme of the functional composition of the example 2-1 of the present invention, the tests were conducted by changing the method disclosed in Chintakunta et al., Eur. J. Med. Chem., 37. pp 339-347, 2002, and the oxidation ratio of (N,N,N′,N′-tetramethyl-p-phenylene-diamine, hereinafter referred to TMPD) was measured while they were reduced from prostaglandin G2(PG G2) to prostaglandin H2(PG H2).

100 mM tris buffer, pH 8.0, 3 um EDTA, 15 uM hematin, 150 unit enzyme(COX-1, COX-2, Sigma corporation, USA, each C-0733, C-0858) were mixed with 1 mL, and the functional composition of the example 2-1 and indomethacin of both control groups were added, and the value obtained by measuring the initial speed that TMPD was oxidized for 25 seconds at 603 nm, and the IC₅₀ value was obtained using the formula 2.

COX-1 or COX-2 inhibition activation(%)=((control group A−control group B)−(sample A−sample B)/control group A−control group B)×100 [Formula 2]

control group A: initial reaction speed of control group not added with sample

Sample A: initial reaction speed of reaction group added with sample

control group B: initial reaction speed of control group not added with Arachidonic acid.

Sample B: initial reaction speed of reaction group not added with Arachidonic acid.

As a result of the tests, the inflammation related enzyme inhibition effects of the functional composition of the example 2-1 had two times less activation(5.57/2.79) as compared to indomethacin of NSAIDs in COX-2, and in COX-1, as being compared with indomethacin, about 730 times less(14.6/0.02) activity was obtained. So, it is considered that it is possible to minimize the side effects of stomach tube and kidney owing to the high inhibition of COX-1, which is the problem of NSAIDs. In fact, when the ratio of COX-2 IC₅₀/COX-1 IC₅₀ is compared with indomethacin, 367 times(139.5/0.38) less effect was obtained. The functional composition of the example 2-1 is known to more selectively inhibit as compared to Indomethacin. (refer to Table 3).

TABLE 3

| | COX IC50(ppm) | | Ratio of COX-2 |
|---|---|---|---|
| | COX-1 | COX-2 | IC50/COX-1 IC50 |
| Functional | 14.6 | 5.57 | 0.38 |
| composition | | | |
| Indomethacin | 0.02 | 2.79 | 139.5 |

Test 4.Activation Inhibition Test of Mast Cell

4-1. Separation Method of Mast Cell

The test was conducted using the method by Chae Ok-hee (effects of bark of mulberry tree to activation of white rat abdomen mast cell based on human, Graduate school report of Junbook national university, 1997). Eight white rats provided from Orient Bio corporation, Mongdong 699-13, Bukmyeon, Gapyung Gun, Kyunggi-do, Korea were anesthetized with ether, and 10 ml of HEPES-Tyrode buffer was injected into the abdomen of white rat, and the abdomen wall was smoothly massaged for 9 seconds. The center of the abdomen was cut, and the abdomen washing liquid was collected using eyedropper, and was sunk for 10 minutes with 200×g, and the supernatant was discarded, and it was floated again until the number of mast cells was 1×106 cell/mL with 10 ml of HEPES-Tyrode buffer. The reliable separation of the mast cells from the floating substance of the abdomen mast cell was conducted using the method by Hachisuka et al (Hachisuki et al. Clinica Chimica Acta, 171, pp 247-256, 1988). A cell floating liquid was placed on isotonic percoll solution(10× Hank's solution 1 mL+percoll 9 mL), and Hepes-Tyrode buffer of 10 mL was filled, and placed for 10 minutes, and the supernatant was discarded by processing for 15 minutes at 125×g, and it was washed two times with Hepes-Tyrode buffer for thereby preparing a pure mast cell floating liquid.

4-2. Cell Toxicity Test

In order to measure the cell toxicity with respect to the functional composition of the example 2-1, abdomen mast cell(2×105 cells in 0.2 mL) was obtained, and only Hepes-Tyrode buffer of 10 mL (25 mL) was processed, and the functional composition(100, 10 or 1 mg/mL) of 25 ml of the example 2-1 with various concentrations were processed and cultivated at 37° C., and the 4-hour cell survival state was checked as follows using Tempkin using a trypan blue and Levi-Schaffer (Temkin and Levi-Schaffer, Cytokine, 15(1), pp 20-26, 2001). The survival ratio was computed assuming that the survival is 100 when abdomen mast cells are obtained.

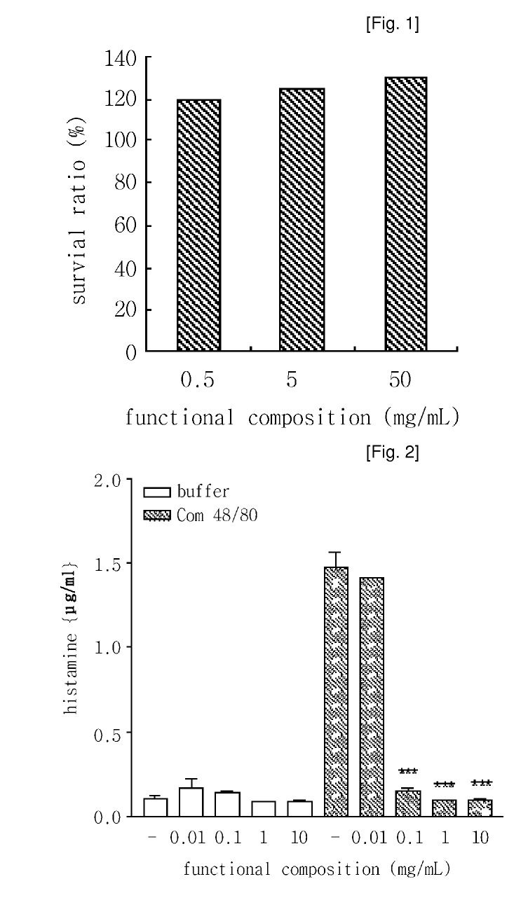

As a result of the test, as shown in FIG. 1, it was shown that the functional composition of the example 2-1 did not have cell toxicity.

4-3. Histamine Separation Measurement from Mast Cells

Locker solution, compound of 48/80 or function or functional solution of 20 ul of the example 2-1 of various concentrations were added to 180 ul of the pure separated mast cell floating liquid (106 cells/ml) in the above-described mast cell separation method, and the amount of histamine from the mast cells was measured. In addition, the histamine separation based on the compound 48/80 was pretreated with a sample solution so as to check the inhibition of the functional composition of the example 2-1, and the amount of histamine was measured by adding the compound 48/80. The amount of histamine was measured using a radioisotope enzymatic assay as follows. (Harvima et al Clin Chim Acta 171 pp 247-256, 1988).

As shown in FIG. 2, as a result of the test, the histamine separated from the white rat abdomen mast cell using the compound 48/80 solution was pre-treated starting from the concentration of the functional composition 0.1 mg/mL of the example 2-1.

4-4. Mast Cell Degranulation Observation and Degranulation Ratio Computation

The abdomen floating liquid of 20 ul with the mast cells that the reaction was finished for checking the type of the mast cell and the degranulation was placed on the slide glass, and was processed at a room temperature for 10 minutes so that the mast cells were precipitated. The mast cells were observed as follows at 1000 times magnitude for thereby separating normal types and degranulation types. Almost abdomen mast cells were filled in circular or egg shapes in the cytoplasm, and the cell boundary was clear. The state that the cytoplasm was filled with the granules is called a normal type mast cell. When the cell boundary was not clear or the granules in the cytoplasm was protruded from the surfaces of the cells or was spread around the cells, they were separated as degranulation. The number of the mast cells observed in the selected 10-vision per a test group was counted, and the degranulation was computed using the formula 3.

Degranulation ratio(%)=(number of mast cells degranulated)/number of mast cells)×100 [Formula 3]

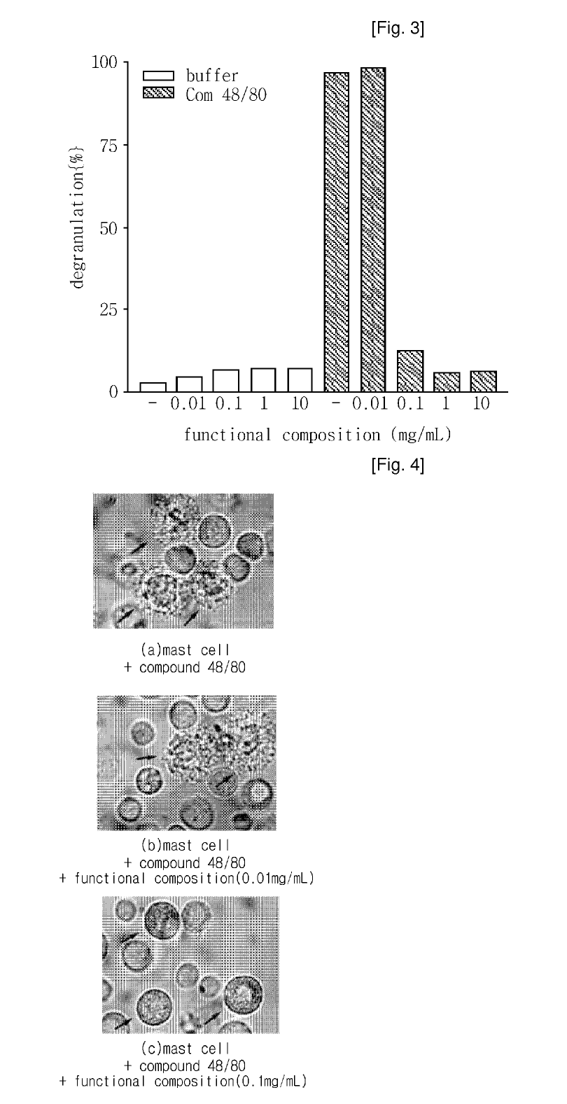

As a result of the test, as shown in FIGS. 3 and 4, the white rat abdomen mast cell degranulation was inhibited from the concentration of the functional composition 0.1 mg/mL of the example 2-1.

Test 5. Passive Cutaneous Anaphylaxis PCA

The following method was conducted so as to know the effectiveness with respect to the functional composition by causing allergic reaction in the actual animal (mouse) with respect to the functional composition of the example 2-1 of the test in the test tube. Anti-DNP (anti-Dinitrophenyl) of 1.2 ug/mL, D8406, Sigma was injected by 10 ul into both ears of a male mouse, and 47 hours after antibody Dinitrophenyl injection, the sample is oral-administrated. One hour later (48 hours after antibody-Dinitrophenyl injection), 1.25 mg of DNP-albumin, A6661, Sigma and a biological salt solution dissolved with 1.25 mg of evans blue, E2129, Sigma was injected into a tail vein for thereby causing a certain reaction. Neck back bone was broken, and the mouse was dead, and the evans blue color component was measured from both ears. As control groups, promethazine hydrochloride, P465, Sigma was used.

As a result of the test, as shown in Table 4, it was known that the allergic reaction improvement effects were obtained from the group that had 5 mg/kg of the functional composition of the example 2-1.

TABLE 4

| | Administration | Amount of Color |
|---|---|---|
| Groups | amount | (ug/a pair of ears |
| Comparison group | — | 17.2 ± 0.8 |
| Functional | 2 | 13.0 ± 1.3 |
| composition | 5 | 9.9 ± 1.4 |
| | 10 | 7.6 ± 0.3 |
| Promethazine | 2 | 7.8 ± 0.5 |
| hydrochloride | 5 | 5.4 ± 1.1 |
| | 10 | 1.7 ± 1.0 |

H1 histamine receptor antagonist

Test 6. Toxicity Test of Animal

10 rats of 8 weeks SD(5 male rats of 250g, and 5 female rats of 210g from Samtoco corporation(Seorang-dong, Osan city, Kyungki-do, Korea) were classified into the control group of 2 rats and the test groups of 8 rats, and 5g/kg concentration was orally administrated one time, and the survival state for two weeks and weight, gloss of hair, upright state, defecation state, and entire mobility were checked.

As a result of the test, 8 rats of the test group administrated with the functional composition of the example 2-1 were all survived, and the health state of the same was good, and a particular matter was not found.

TABLE 5

| | | weight | | | |
|---|---|---|---|---|---|
| | | Initial | After one | After 2 | Health |
| Groups | Sex | stage | week | weeks | state |
| Test group | Female | 237 | 288 | 301 | Good |
| | | 221 | 287 | 314 | Good |
| | | 231 | 295 | 301 | Good |
| | | 234 | 283 | 321 | Good |
| Comparison | | 222 | 281 | 319 | Good |
| group | | | | | |
| Test group | male | 181 | 201 | 217 | Good |
| | | 189 | 207 | 219 | Good |
| | | 187 | 211 | 238 | Good |
| | | 191 | 216 | 234 | Good |
| Comparison | | 190 | 211 | 241 | good |
| group | | | | | |

Test 7. Clinic Test

A temporal clinic test was conducted in such a manner that 39 patients suffering from allergic rhinitis were administrated by 400 mg(two pills per one time, two times a day) in four hospitals so as to check the allergic rhinitis effects of the human body with respect to the functional composition of the example 2-1 of the present invention (refer to Table 6).

As a result of the above test, 32 patient had improvements among 39 patients without side effects, and 7 patients including server 3 patients had not improvements.

TABLE 6

| Name of | Days of | | | Number of | |
|---|---|---|---|---|---|
| hospital | administration | Sex | ages | persons | Contents |
| Hangang | 15 days | Male | 30 | 2 | No side effects to all |
| Sumshim | | Male | 40 | 4 | patients, and allergic |
| otolaryngology | | female | 30 | 4 | rhinitis was improved 1 |
| | | female | 40 | 2 | week after administration |
| Mirae | 30 days | Male | 10 | 3 | All patients improved |
| otolaryngology | | Male | 30 | 3 | without side effects |
| | | Female | 10 | 2 | |
| | | Female | 20 | 1 | |
| | | Female | 40 | 2 | |
| Jang Pyunha | 20 days | Male | 20 | 2 | No side effects were |
| internal | | Male | 30 | 3 | found, allergic rhinitis |
| medicine | | Male | 40 | 1 | such as sneeze and cough |
| | | Female | 20 | 2 | were improved except in |
| | | Female | 30 | 2 | 2 females in their 20s, |
| | | Female | 40 | 1 | and 2 females in their 30s |
| Shim Sangryul | 25~30 days | Male | 30 | 2(1 person | Light patients symptom were |
| otolaryngology | | female | 30 | intense) | improved, and intense |
| | | | | 3(2 persons | patients were not |
| | | | | intense) | improved. |

The formation of the medicine composition including the combined crude drug extract was described according to the present invention. But it was not limited thereto. Only the detailed description was provided.

### Formulation Example 1

### Preparation of Acids

Functional composition of example 2-1 30 mg

Lactose 100 mg

Talc 10 mg

Acids are prepared by mixing the above components and filling a sealing fabric.

### Formulation Example 2

### Preparation of Pills

Functional composition of example 2-2 50 mg

Starch of corn 100 mg

Lactose 100 mg

Stearin acid magnesium 2 mg

The above components are mixed, and the formulation is manufactured based on the pill preparation method.

### Formulation Example 3

### Preparation of Capsule

Functional composition of example 2-1 50 mg

Starch of corn 100 mg

Lactose 100 mg

Stearin acid magnesium 2 mg

The above components are mixed based on a conventional capsule agent preparation method, and it is filled in a gelatin capsule, and the capsule product is manufactured.

### Formulation Example 4

### Preparation of Injection Agent

Functional composition of example 2-2 50 mg

Disinfected distilled water for injection a proper amount

PH adjuster a proper amount

It is manufactured with the above component amount 2 ml per one ample based on a conventional manufacturing method of injection material.

### Formulation Example 5

### Preparation of Liquid Agent

Functional composition of example 2-1 100 mg

Isomerized Sugar 10 g

Manitol 5g

Distilled water a proper amount

Each component of distilled water is added and dissolved based on the preparation method of liquid agent, and a lemon aromatic is added and mixed with the above component, and the entire mixture is added with distilled water for thereby adjusting to 100 ml, and it is filled in a brown color bottle and is disinfected for thereby preparing liquid agent.

### Formulation Example 6

### Preparation of Health Food

Functional composition of example 2-1 100 mg

Vitamin mixture a proper amount

Vitamin A acetate 70 um

Vitamin E1.0 mg

Vitamin B11.13 mg

Vitamin B2 1.15 mg

Vitamin B6 0.5 mg

Vitamin B12 0.2 ug

Vitamin C 10 mg

Biotin 10 ug

nicotinic acid amide 1.7 mg

Folic acid 50 ug

Pantothenic Acid calcium 0.5 mg

Inorganic mixture a proper amount

Ferrous Sulphate 1.75 mg

Zinc oxide 0.82 mg

Carbonic acid magnesium 25.3 mg

Single valued potassium phosphate 15 mg

Double valued phosphoric calcium 55 mg

Citric potassium 90 mg

Calcium carbonate 100 mg

Chloride magnesium 24.8 mg

The composition ratio of the vitamin and mineral mixture is mixed with a certain component proper to the health food. The mixing ratio may be changed randomly, and the above components are mixed based on a conventional health food preparation method, and the granule is manufactured, and the present invention may be used for the health food manufacturing method.

### Formulation Example 7

### Preparation of Health Beverage

Functional composition of example 2-2 1000 mg

Citric acid 1000 mg

Oligosaccharide 100g

Concentrated liquid of Japanese apricot tree fruit 2g

Taurine 1g

Entire portions added with distilled water 900 ml

The components are mixed based on the conventional health beverage preparation method and are agitated for about 1 hour at 85° C., and the prepared solution is stored in the container of 21 by filtering the same. It is disinfected and sealed for thereby preparing the health beverage composition of the present invention.

The composition is determined with the components proper to a relatively favorite beverage, and the mixing ration may be changed based on the demand class, needed countries, the purpose of use and place and national characteristic.

### INDUSTRIAL APPLICABILITY

As described above, the functional composition of the present invention may be used for manufacturing a medicine composition and health function food with its anti-allergic rhinitis, anti-atopic dermatitis and anti-asthma activity effects.

1. A prevention and therapy pharmacy composition for allergic rhinitis, atopic dermatitis and asthma which includes at least one crude drug extract selected from the group including wormwood, guava leaves, mulberry tree leaves and grape seeds as an effective component.

2. The composition of claim 1, wherein said extract is obtained from a combined crude drug extract of wormwood, guava leaves, mulberry tree leaves and grape seeds.

3. The composition of claim 2, wherein said mixing ratio of the combined crude drug extract is 1˜3 weight % of wormwood, 1˜2 weight % of guava leaves, 1˜2 weight % of mulberry tree leaves, and 2˜4 weight % of grape seeds.

4. A health function food for a prevention and improvement of allergic rhinitis, atopic dermatitis and asthma which includes at least one crude drug extract selected from the group including wormwood, guava leaves, mulberry tree leaves and grape seeds as an effective component.

5. The food of claim 4, wherein said extract is a combined crude drug extract of wormwood, guava leaves, mulberry tree leaves and grape seeds.

6. The food of claim 5, wherein said mixing ratio of the combined crude drug extract is 1˜3 weight % of wormwood, 1˜2 weight % of mulberry tree leaves, 1˜2 weight % of guava leaves, and 2˜4 weight % of grape seeds.

7. The food of claim 4, wherein said food is a formulated capsule pill or a liquid health function food.
